(19) United States
(12) Patent Application Publication
(10) Pub. No.: US 2010/0291698 A1
(43) Pub. Date: Nov. 18, 2010

(54) DETECTION OF NITRO- AND NITRATE-CONTAINING COMPOUNDS

(75) Inventors: William C. Trogler, Del Mar, CA (US); Jason Sanchez, San Diego, CA (US); Sara Toal, Mission Viejo, CA (US); Zheng Wang, La Jolla, CA (US); Regina E. Dugan, Rockville, MD (US)

Correspondence Address: GREER, BURNS & CRAIN, 300 S WACKER DR, 25TH FLOOR, CHICAGO, IL, 60606, US

(73) Assignee: THE REGENTS OF THE UNIVERSITY OF CALIFORNIA, Oakland, CA (US)

(21) Appl. No.: 12/298,075

(22) PCT Filed: May 1, 2007
(86) PCT No.: PCT/US07/10583

§ 371 Date: May 5, 2010

Related U.S. Application Data
(60) Provisional application No. 60/797,328, filed on May 3, 2006.

(51) Int. Cl. G01N 33/52

(52) U.S. Cl. 436/110; 436/106; 436/111

(57) ABSTRACT

A method of the invention is a method of detecting nitramines and nitrate esters believed to be present on a sampling substrate. In the method, a sampling substrate is exposed to a first reagent that is formulated to react with nitramine- and nitrate ester-type explosives to release nitrite. The sampling substrate is then exposed to a second reagent that contains an acid to react with the nitrite and a diaminoaromatic present in either the first or second reagent, to form a triazole that will luminesce. Another method of the invention combines this process for nitramine- and nitrate ester-based explosives detection with a technique to detect nitroaromatic-based explosives using luminescent polymers, for a three-step process for the detection of explosives in these three classes.

### PRIORITY CLAIM

This application claims priority under 35 U.S.C. 119 from prior provisional application Ser. No. 60/797,328, which was filed on May 3, 2006.

### STATEMENT OF GOVERNMENT INTEREST

This invention was made with government assistance from the U.S. Air Force Office of Scientific Research Contract #AFOSR F49620-02-0288. The Government has certain rights in this invention.

### TECHNICAL FIELD

A field of the invention is analyte detection. The present invention is directed to inorganic polymers and use of inorganic polymers, namely luminescent metallole-containing polymers and copolymers, including photoluminescent or electroluminescent polymers, and/or the use of diaminoaromatics, for detection of organic nitrogen-based explosives.

### BACKGROUND ART

Use of chemical sensors to detect ultra-trace amounts of explosives and explosive-related analytes has been the focus of investigation in recent years owing to the critical importance of detecting explosives in a wide variety of areas, such as mine fields, military bases, remediation sites, and urban transportation areas. Detecting explosive analytes also has obvious applications for homeland security and forensic applications as well. Low-cost chemical sensors that utilize simple colorimetric or synthetic polymer/molecules to provide a measurable signal, in particular an easily observed or transduced optical signal upon interaction with specific analytes, are highly desired.

Chemical sensors are preferable to other detection devices, such as metal detectors, because metal detectors frequently fail to detect explosives, such as those in the case of the plastic casing of modern land mines. Similarly, trained dogs can be both expensive and difficult to maintain in many desired applications. Other detection methods, such as gas chromatography coupled with a mass spectrometer, surface-enhanced Raman, nuclear quadrupole resonance, energy-dispersive X-ray diffraction, neutron activation analysis and electron capture detection are highly selective, but are expensive and not easily adapted to a small, low-power package.

Conventional chemical sensors have drawbacks as well. Sensing TNT and picric acid in groundwater or seawater is important for the detection of buried, unexploded ordnance and for locating underwater mines, but most chemical sensor detection methods are only applicable to air samples because interference problems are encountered in complex aqueous media. Thus, conventional chemical sensors are inefficient in environmental applications for characterizing soil and groundwater contaminated with toxic TNT at military bases and munitions production and distribution facilities. Also, conventional chemical sensors, such as n-conjugated, porous organic polymers, can be used to detect vapors of electron deficient chemicals, but require many steps to synthesize and are not selective to explosives.

Furthermore, many conventional chemical sensing methods are not amenable to incorporation in inexpensive, low-power portable devices. Additionally, these methods are limited to vapor phase detection, which is disadvantageous given the low volatility of many explosives. For example, the vapor pressure of TNT, which is approximately 5 ppb at room temperature, may result in vapor concentrations up to six times lower when enclosed in a bomb or mine casing, or when present in a mixture with other explosives.

Lastly, the broad array of nitrogen-based explosives renders it difficult to provide a single method whereby multiple types of explosives may be detected.

### DISCLOSURE OF INVENTION

A method of the invention is a method of detecting nitramines and nitrate esters believed to be present on a sampling substrate. In the method, a sampling substrate is exposed to a first reagent that is formulated to react with nitramine and nitrate ester type explosives to release nitrite. The sampling substrate is then exposed to a second, reagent that contains an acid to react with the nitrite and a diaminoaromatic, present in either the first, second or third reagent, to form a triazole that will luminesce under exposure to a stimulation wavelength.

In another method of detecting of the invention, the presence or absence of nitroaromatic-based explosives is initially determined using luminescent polymers and copolymers to observe fluorescence quenching by nitroaromatic-based explosives. The luminescent polymers and copolymers include photoluminescent or electroluminescent polymers. The luminescence of the polymers and copolymers is then eliminated under alkaline conditions, and then the presence or absence of either nitrate ester- or nitramine-based explosives is determined by observing the presence or absence of luminescence from a triazole compound.

Another method for detecting of the invention detects one or more nitrogen-based explosives that may be present in a sampling substrate or in an environment to which the sampling substrate has been exposed. The sampling substrate is exposed to a first reagent having a luminescent polymer or copolymer to detect nitroaromatic explosive particulates. The sampling substrate is then exposed to a stimulation wavelength, and the presence or absence of luminescence is observed to determine the corresponding presence or absence of nitroaromatic explosive particulates. The sampling substrate is exposed to a second reagent capable of both degrading the luminescent polymers of the first reagent, and reacting with nitramine and nitrate ester type explosives to release nitrite. A third reagent is reacted with the nitrite and a diaminoaromatic also present in one of either the first, second or third reagent to form a luminescent compound. The sampling substrate is again exposed to a stimulation wavelength, and the presence or absence of stimulated luminescence is observed to determine the corresponding presence or absence of nitrate ester or nitramine based explosives.

### BRIEF DESCRIPTION OF THE DRAWINGS

FIG. 1 is a model of a polysilole molecule;

FIG. 2 illustrates a pair of equations for the synthesis of polygermole and polysilole according to an embodiment of the invention;

FIG. 3 illustrates a pair of equations for the synthesis of a silole-germole copolymer according to an embodiment of the invention;

FIG. 4 illustrates a pair of equations for the synthesis of silole-silane alternating copolymers according to an embodiment of the invention;

FIG. 5 is a table of the absorption and fluorescence spectra observed in one embodiment of the invention and taken at the concentrations of 2 mg/L in THF and 10 mg/L in toluene, respectively;

FIG. 6 is a schematic energy level diagram illustrating energy-levels for polymetalloles and metallole-silane copolymers;

FIG. 7 is a graphical representation of UV-vis absorption spectra in THF (solid line) and fluorescence spectra in toluene (dotted line) for (A) poly(tetraphenyl) germole 2. (B) silole-silane copolymer 4, and (C) germole-silane copolymer 9;

FIGS. 8A and 8B illustrate a HOMO (A) and LUMO (B) of 2.5-diphenylsilole, Ph₂C₄SiH₂ from the ab initio calculations at the HF/6-31G* level;

FIG. 9 is a graphical representation of the fluorescence spectra of polysilole 1 in toluene solution (solid line) and in thin solid film (dotted line);

FIG. 10 is a graphical representation of the quenching of photoluminescence spectra of silole-silane copolymer 5 with (A) nitrobenzene, from top 2.0×10-5 M; 3.9×10⁻⁵ M, 7.8×10⁻⁵ M, and 11.5×10⁻⁵ M, (B) DNT, from top 1.4×10⁻⁵ M, 3.9×10⁻⁵ M, 7.8×10⁻⁵ M, and 12.4×10⁻⁵ M, (C) TNT, from top 2.1×10⁻⁵ M, 4.2×10⁻⁵ M, 8.1×10⁻⁵ M, and 12.6×10⁻⁵ M, (D) picric acid, from top 2.1×10⁻⁵ M, 4.2×10⁻⁵ M, 8.0×10⁻⁵ M, and 12.6×10⁻⁵ M;

FIGS. 11A, 11B and 11C are Stern-Volmer plots; from top polysilole 1, polygermole 2, and silole-silane copolymer 8; [symbol] (picric acid), ▪ (TNT), ♦ (DNT), (nitrobenzene); the plots of fluorescence lifetime (τₒ/τ), shown as inset, are independent of added TNT;

FIG. 12 illustrates fluorescence decays of polysilole 1 for different concentrations of TNT: 0 M, 4.24×10⁻⁵ M, 9.09×10⁻⁵ M, 1.82×10⁻⁴ M;

FIG. 13 illustrates Stern-Volmer plots of polymers [symbol] (polymer 1), ▪ (polymer 5), ♦ (polymer 4), (polymer 6), ¤ (polymer 2), and — (organic pentiptycene-derived polymer 13), for TNT;

FIG. 14 illustrates a structure of the pentiptycene-derived polymer;

FIG. 15 illustrates, from left to right, highest and lowest photoluminescence quenching efficiency for picric acid (left-most two lines), TNT (two lines immediately to the right of picric acid), DNT (two lines immediately to the right of TNT), and nitrobenzene (right-most two lines) showing how the varying polymer response to analyte could be used to distinguish analytes from each other;

FIG. 16 illustrates a comparison of the photoluminescence quenching constants (from Stem-Volmer plots) of polymers 1-12 with different nitroaromatic analytes;

FIG. 17 illustrates a plot of log K vs reduction potential of analytes: [symbol] (polymer 1), ▪ (polymer 2), ♦ (polymer 3), (polymer 4), ¤ (polymer 5), and (polymer 10);

FIG. 18 illustrates a schematic diagram of electron-transfer mechanism for quenching the photoluminescence of polymetallole by analyte;

FIG. 19 illustrates an absence of quenching of photoluminescence by polysilole 1 with 4 parts per hundred of THE; and

FIG. 20 illustrates an equation for a catalytic dehydrocoupling method for synthesizing metallole polymers according to one embodiment of the invention.

FIGS. 21a, 21b and 21c illustrate various copolymers as well as their syntheses, namely PDEBSi, PDEBGe, PDEBSF, PDEBGF, PSF and PGF; and

FIG. 22 is a table summarizing the detection limits of TNT, DNT, and picric acid using the five metallole-containing polymers synthesized, PSi, PDEBSi, PGe, PDEBGe, and PDEBSF.

### BEST MODE FOR CARRYING OUT THE INVENTION

While efficient explosives detection has always been a predominating concern, there exists a renewed urgency for development of rapid and highly sensitive detection of organic, nitrogen-based explosives, including nitroaromatic-based, nitramine-based, and nitrate ester-based explosives. In addition to detecting TNT, for example, detection of the nitrogen-based plastic explosives compounds associated with improvised explosives devices (IEDs), such as RDX (Cyclotrimethylenetrinitramine) and PETN (Pentaerythritol Tetranitrate) and military explosive compositions containing these explosives, such as C4, has life-saving implications in a vast array of applications, such as, military, and civilian homeland security purposes.

Accordingly, while previous work has provided methods and sensors useful for detecting trace quantities of nitroaromatic compounds, embodiments of the present invention are especially advantageous in providing methods and sensors for detecting trace quantities of additional organic, nitrogen-based explosives, such as nitrate esters and nitramine-based explosives.

Various embodiments of the invention provide sensors and sensing methods for detecting, through one or more steps, trace residues of one or more solid state explosives. Embodiments include methods for detection of nitramine- and nitrate ester-based explosives using ortho-diaminoaromatic compounds to form a luminescent triazole compound. Other embodiments include methods for detection of all three classes of nitroaromatic-based, nitramine-based and nitrate ester-based explosives using 1) luminescence quenching of luminescent polymers to detect nitroaromatic-based explosives, and 2) nitramine- and nitrate ester-based explosives detection through a two-step process that forms a luminescent triazole compound. Embodiments of the invention are particularly advantageous in that the methods and sensors are sensitive, rapid, low-cost, and capable of detecting a wide range of trace explosives from or on a variety of surfaces, including bomb makers' hands, clothing, hair, dwellings, packages, cars, and door knobs to their houses, to name a few.

Various embodiments of the invention exploit advantageous properties of luminescent metallole polymers and copolymers, e.g., photoluminescent or electroluminescent polymers. Luminescent metallole polymers are stable in air, water, acids, common organic solvents, and even seawater containing bioorganisms.

Metalloles are silicon (Si) or germanium (Ge) containing metallocyclopentadienes. Silole and germole dianions (RC)₄Si²⁻ and (RC)₄Ge²⁻, where R=Ph or Me, have been studied by X-ray crystallography and found to be extensively delocalized. Siloles and germoles are of special interest because of their unusual electronic and optical properties, and because of their possible application as electron transporting materials in devices. Polysilanes and polygermanes containing a metal-metal backbone emit in the near UV spectral region, exhibit high hole mobility, and show high nonlinear optical susceptibility, which makes them efficient emission candidates for a variety of optoelectronics applications. These properties arise from a σ-σ* delocalization along the M-M backbones and confinement of the conjugated electrons along the backbone.

Polymetalloles and metallole-silane copolymers are unique in having a Si—Si, Ge—Ge, or Si—Ge backbone encapsulated by the highly conjugated unsaturated five-membered ring systems as side chains. These polymers are highly luminescent, and are accordingly useful in light-emitting-diode (LED) applications and as chemical sensors. Characteristic features of polymetalloles and metallole-silane copolymers include a low reduction potential and a low-lying lowest unoccupied molecular orbital (LUMO) due σ*-π* conjugation arising from the interaction between the σ* orbital of silicon or germanium and the π* orbital of the butadiene moiety of the five membered ring. In addition, the M-M backbones exhibit σ-σ* delocalization, which further delocalizes the conjugated metallole π electrons along the backbone. Electron delocalization in these polymers provides a means of amplification, because interaction between an analyte molecule at any position along the polymer chain is communicated throughout the delocalized chain.

More particularly, embodiments of the present invention provide a rapid, low cost, highly sensitive method of detection for a range of explosive materials including nitroaromatic-, nitrate ester-, and nitramine-based explosives. In one exemplary method, a sampling substrate is sequentially exposed to a plurality of detection reagents, preferably three reagents, to determine the presence and amount of various solid explosive particulates.

While the sampling substrate may be separate from the surface suspected of being contaminated with the target explosive, i.e., a substrate exposed to a potentially contaminated surface, the sampling substrate may also include the contaminated surface itself. One exemplary sampling substrate is filter paper that is contacted with, or otherwise exposed to, the contaminated surface. Generally, the sampling substrate can be a surface or environment that is suspected of being contaminated.

A method of the invention is a method of detecting nitramines and nitrate esters believed to be present on a sampling substrate. In the method, a sampling substrate is exposed to a first reagent that is formulated to react with nitramine and nitrate ester explosives to release nitrite. The sampling substrate is then exposed to a second reagent that contains an acid to react with the nitrite and a diaminoaromatic, present in either the first or second reagent, to form a triazole that will fluoresce under exposure to a stimulation wavelength.

In another method of detecting of the invention, the presence or absence of nitroaromatic-based explosives is determined using photoluminescent polymers and copolymers to observe fluorescence quenching by the nitroaromatic-based explosives. Luminescence, e.g., photoluminescence, of the luminescent metallole polymers and copolymers is eliminated, then the presence or absence of either nitrate ester- or nitramine-based explosives is determined by observing the presence or absence of fluorescence from a triazole compound.

Another method for detecting of the invention detects one or more nitrogen-based explosives that may be present in a sampling substrate or in an environment to which the sampling substrate has been exposed. The sampling substrate is exposed to a first reagent having a luminescent polymer or copolymer to detect nitroaromatic explosive particulates. The sampling substrate is then exposed to a stimulation wavelength, and the presence or absence of luminescence is observed to determine the corresponding presence or absence of nitroaromatic explosive particulates. The sampling substrate is exposed to a second reagent capable of both degrading the luminescent polymers of the first reagent, and reacting with nitramine and nitrate ester type explosives to release nitrite. A third reagent is reacted with the nitrite and a diaminoaromatic also present in one of either the first or second reagent to form a luminescent compound. The sampling substrate is exposed to a stimulation wavelength, and the presence or absence of stimulated fluorescence is observed to determine the corresponding presence or absence of nitrate ester- or nitramine-based explosives.

Preferred embodiment detection methods will now be discussed with reference to the drawings. Testing results are included herein, and broader aspects of the invention and additional features will be apparent to artisans from the preferred embodiment description and the testing results.

In a preferred embodiment method of detection, a first detection step detects even extremely small amounts of nitroaromatic-based explosives, in low nanogram quantities. Nitroaromatic-based explosives detected in the first step include, for example, trace residues of picric acid (PA, 2,4,6-trinitrophenol, C₆H₂(NO₂)₃OH), nitrobenzene (NB, C₆H₅NO₂), 2,4-dinitrotoluene (DNT, C₇H₆N₂O₄) and 2,4,6-trinitrotoluene (TNT, C₇H₅N₃O₆).

In the first detection step, the sampling substrate is first exposed to a first reagent, Reagent A. Reagent A is preferably selected for properties contributing to detection of nitroaromatic explosives, such as TNT, DNT, tetryl and picric acid, on the sampling substrate. Based on experimental results, it is predicted that Reagent A may include one of a variety of volatile organic solvents and one of a variety of luminescent polymers. While a broad array of luminescent polymers are contemplated for use with the invention, exemplary luminescent polymers include photoluminescent metallole-containing polymers, polyacetylenes, poly(p-phenylenevinylenes), and poly(p-phenyleneethynylenes).

One of a variety of diamino aromatic compounds, such as 2,3-diaminonapthalene, 1,8-diaminonapthalene, 9,10-diaminophenanthrene, or 1,2-diaminoanthraquinone, may also be added in Reagent A for subsequent reactions with Reagent B and C to detect nitramine- and nitrate ester-based explosives. Preferably, Reagent A includes a silole or germole (metallole) luminescent polymer or metallole-containing copolymer. Metalloles and metallole copolymers have the advantage of being inexpensive and easily prepared. However, other photoluminescent polymers such as polyacetylenes, poly(p-phenyleneethynylenes), and poly(p-phenylenevinylenes) may also be used in the method. In addition, electroluminescent polymers can be used.

Specifically, Reagent A preferably includes at least one of a Polysilole, Polygermole, Poly(1,4-diethynylbenzene)2,3,4,5-tetraphenylsilole (PDEBsilole), Poly(1,4-diethynylbenzene)2,3,4,5-tetraphenylgermole (PDEBgermole), Poly(1,4-diethynylbenzene)silafluorene (PDEBSF), Poly(1,4-diethynylbenzene)germafluorene (PDEBGF), Polysilafluorene (PSF) and Polygermafluorene (PGF).

For purposes of discussion only, one exemplary Reagent A includes a 1 mg/mL solution of poly(tetraphenyl)silole in a 1:1 acetone:toluene solvent. Prior to use with embodiments of the invention, Reagent A is preferably stored in degassed or deoxygenated solvents and is protected from UV exposure to preserve the polymer from oxidation and/or photodegredation. Other volatile solvents, luminescent polymers, and concentrations are expected to work in the method.

Reagent A is sprayed on or otherwise deposited on the sampling surface. In a preferred method, each of Reagents A, B, and C are sprayed onto the sample substrate at a volumetric flow rate of approximately 0.5 mL/s. The sampling substrate and Reagent A are then excited at an appropriate wavelength, such as 360 nm, with a blacklight, LED, or other illumination source. Detection of nitroaromatic explosives such as TNT, DNT, and picric acid is confirmed by visually or instrumentally (e.g. with a U.V. or visible CCD camera, or a fluorimeter) detecting quenching of the fluorescence emission of the polymer (Reagent A) by the analyte (e.g., TNT, DNT and picric acid). Advantageously, detection is selective for the strongly oxidizing explosives.

After results have been obtained from exposure of the sampling substrate to Reagent A, the sampling substrate, which is already in contact with Reagent A, is sprayed with or otherwise exposed to Reagent B.

Reagent B may be selected such that the metallole polymer or other luminescent polymer from Reagent A is destroyed through degradation of the polymer, usually through degradation of the polymer backbone, thereby eliminating fluorescing properties of the polymer. This reduces or eradicates any background fluorescence, which could subsequently interfere with the explosives detection upon exposing the sampling substrate to Reagent C.

One exemplary Reagent B includes a solution of 2,3-diaminonaphthalene (DAN) (0.6 mg/mL) in a 0.75 M potassium hydroxide (KOH) solution of a 2:9:9 dimethylsulfoxide:acetone:ethanol solvent mixture. Reagent B may reasonably be expected to include other diaminoaromatic compounds, solvents and bases. Prior to use with embodiments of the invention, Reagent B is preferably stored in a dark environment to preserve its contents.

Reagent B is applied to or otherwise deposited on the sampling substrate already having Reagent A disposed thereon. The substrate is then preferably, though optionally, heated with a heat gun or other heat source above a predetermined temperature for a predetermined period of time, such as 90° C. for approximately 1-3 seconds, sufficient to destroy the polymer from Reagent A and also to effectively release nitrite from nitramine or nitrate ester type explosives such as RDX, HMX, nitroglycerine, PETN and tetryl, produced according to the elimination reaction seen in Scheme 1 shown below.

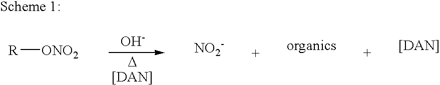

Following the heating step, Reagent C is sprayed on or otherwise deposited on the sampling substrate.

Reagent C is reacted with a nitrite, as well as with a diaminoaromatic that is present in either Reagent A, B or C, to form a luminescent compound, such as 1-H-napthatriazole, which luminesces to indicate the presence of a nitrate ester- or nitramine-based explosive. One preferred Reagent C is selected to have an acid component to react with nitrite to form nitrous acid, which then reacts with the present 2,3-diaminonapthalene (DAN) to form 1-H-napthotriazole according to Scheme 2 below.

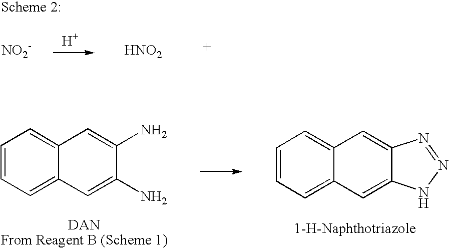

Following application of Reagent C, the sampling substrate is again preferably, though optionally, heated. Heating the sampling substrate after the application of Reagent C helps to speed the reaction as well as to assist in solvent evaporation. When placed under a 360 nm UV lamp, 1-H-naphthotriazole emits blue or greenish-blue fluorescence, which confirms the presence of nitrate ester or nitramine based explosives. Nanogram-level detection limits have been observed visually (observing visible wavelengths) and improved detection may reasonably be expected with UV imaging equipment (increased sensitivity observing UV wavelengths).

One exemplary Reagent C includes a 1:1 solution of phosphoric acid and ethanol. Other acids and organic solvents, such as acetone, are expected to work as well in the acidification step.

In an alternative preferred embodiment, Reagent A includes a 0.5 mg/mL poly(tetraphenyl)siloel and 1 mg/mL 2,3-diaminonaphthalene (DAN) acetone solution. The solution is preferably stored away from UV light to prevent photodegradation. Reagent B includes a 0.75 M KOH solution in 3:2 ethanol:dimethylsulfoxide, though other bases in suitable solvents are expected to work as well. A small amount of water (˜5%) may be added to assist in KOH solubility and solution stability. Reagent C may include the same solutions discussed in the first preferred embodiment, such as the 1:1 solution of phosphoric acid and ethanol or other acids and organic solvents.

Thus, in the alternative preferred embodiment, Reagent A includes DAN, or other diaminoaromatic. Reagent B includes a base (e.g., KOH) that reacts with both nitramine- and nitrate ester-based explosives. Reagent C reacts with the products produced upon reaction of Reagent B with the explosives, which in turn react with the DAN of Reagent A to reveal, via blue or greenish-blue fluorescence, the presence of a triazole, indicating the presence of nitramine- and/or nitrate ester-based explosives.

It is further contemplated that the sampling substrate may be provided with one or more of the Reagents A, B and C already disposed thereon in predetermined regions, where the predetermined regions may assume a variety of geometric configurations, such as each being confined to a stripe of the sampling substrate. With the Reagents A, B and C disposed on a sampling substrate, the sampling substrate may then be exposed to an environment believed to be contaminated by explosives, such that the respective reactions will occur as the explosives contact the respective reagents disposed on the sampling substrate.

Similarly, it is contemplated that a sampling substrate may undergo generally simultaneous application of Reagents A, B and C to predetermined regions following exposure of the sampling substrate to an environment believed to be contaminated by explosives, such that the respect reactions will occur as the respective reagents are applied to the sampling substrate having the explosives already disposed thereon.

### Detection of Nitroaromatic-Based Compounds

In the first step, detection of the nitroaromatic-based explosives may be accomplished by measurement of the quenching of luminescence of luminescent polymers by the analyte. A plot of log K, the Stern-Volmer constant for quenching efficiency of an analyte and fluorophore, versus the reduction potential of analytes (NB, DNT, and TNT) for each metallole copolymer yields a linear relationship, indicating that the mechanism of quenching is attributable to electron transfer from the excited metallole copolymers to the lowest unoccupied orbital of the analyte.

Excitation may be achieved with electrical or optical stimulation. If optical stimulation is used, a light source containing energy that is higher than the energy of emission of the polymer is preferably used. This could be achieved with, for example, a mercury lamp, a blue light emitting diode, or an ultraviolet light emitting diode.

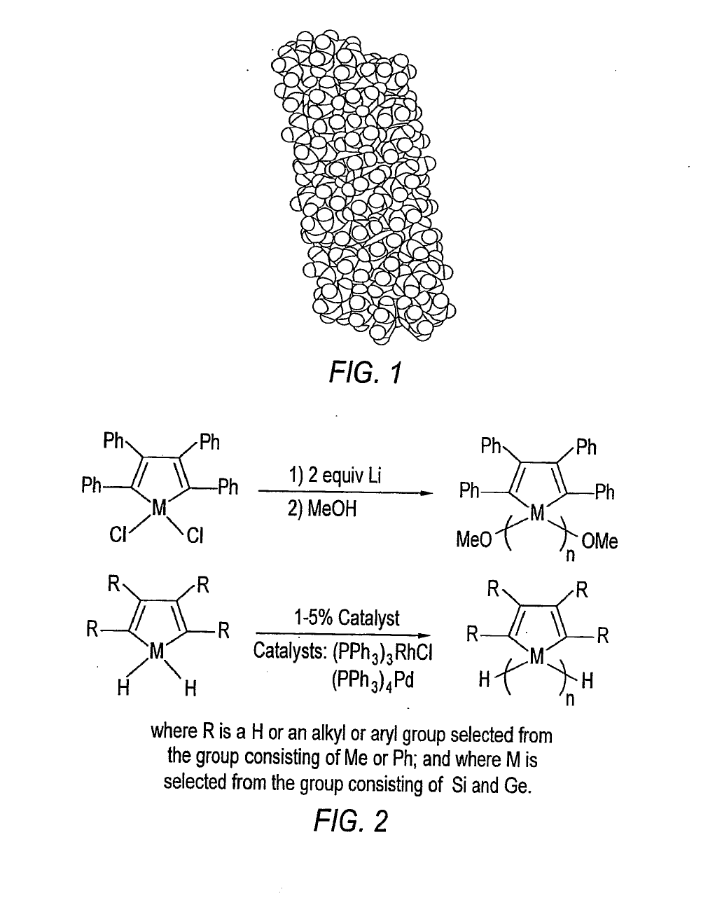

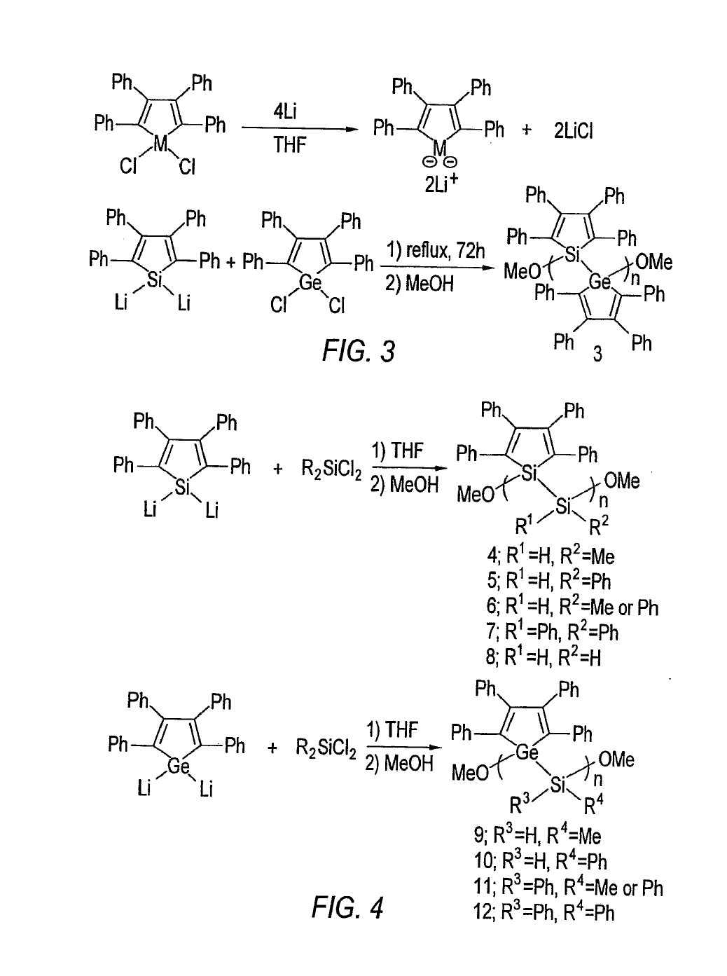

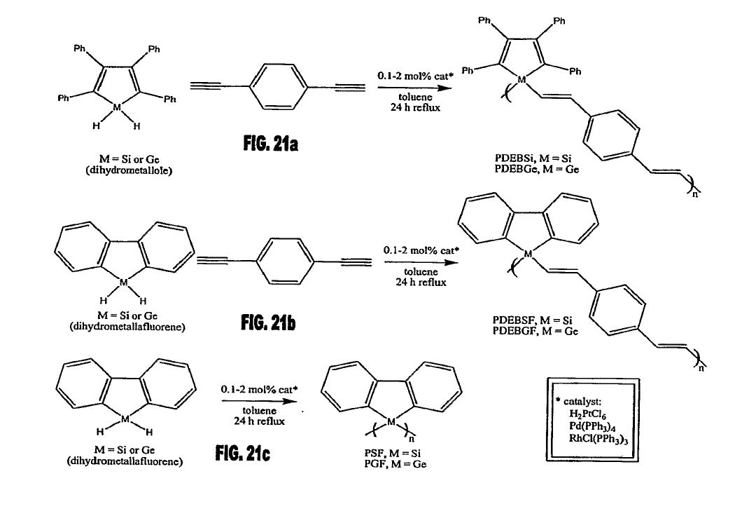

FIG. 1 illustrates a space filling model structure of polysilole 1, which features a Si—Si backbone inside a conjugated ring system of side chains closely packed to yield a helical arrangement. FIG. 2 illustrates polymers 1 and 2, FIG. 3 illustrates polymer 3, and FIG. 4 illustrates copolymers 4-12. FIGS. 21a through 21c illustrate additional copolymers as well as their syntheses, Poly(1,4-diethynylbenzene)2,3,4,5-tetraphenylsilole (PDEBsilole), Poly(1,4-diethynylbenzene)2,3,4,5-tetraphenylgermole (PDEBgermole), Poly(1,4-diethynylbenzene)silafluorene (PDEBSF), Poly(1,4-diethynylbenzene)germafluorene (PDEBGF), Polysilafluorene (PSF) and Polygermafluorene (PGF). A conventional method for preparing polymetalloles and metallole copolymers is Wurtz-type polycondensation. The syntheses of polygermole and polysiloles, and other copolymers are analogous to one another, as illustrated in equation 1 in FIG. 2, and employ the Wurtz-type polycondensation. However, yields from this method of synthesis are low (ca. ˜30%). Thus, Wurtz-type polycondensation is not well-suited to large-scale production.

Catalytic dehydrocoupling of dihydrosiloles with a catalyst is an attractive alternative to Wurtz-type polycondensation. Bis(cyclopentadienyl) complexes of Group 4 have been extensively studied and shown to catalyze the dehydrocoupling of hydrosilanes to polysilanes for the formation of Si—Si bonds. However, only the primary organosilanes react to give polysilane. Secondary and tertiary silanes give dimers or oligomers in low yield. It has been reported that the reactivity decreases dramatically with increasing substitution at the silicon atom, since reactions catalyzed by metallocenes are typically very sensitive to steric effects. Mechanisms for dehydrogenative coupling of silanes have also been extensively investigated, which involves σ-bond metathesis.

One such synthesis utilizes the catalytic dehydrocoupling polycondensation of dihydro(tetraphenyl)silole or dihydro(tetraphenyl)germole with 1-5 mol % of Wilkinson's catalyst, Rh(PPh₃)₃Cl, or Pd(PPh₃)₄, as illustrated in FIG. 2, or 0.1-0.5 mol % of H₂PtCl₆.xH₂O in conjuction with 2-5 equivalents of allylamine, or other alkene, such as cyclohexene, for example, as illustrated in FIG. 20. The latter reactions produce the respective polysilole or polygermole in high yield (ca. 80-90%). By ¹H NMR spectroscopy, the monomer, dihydrometallole, was completely consumed in the reaction. Molecular weights (M_{w}) of 4000-6000 are obtained, similar to those obtained by the Wurtz-type polycondensation (ca. ˜30%).

The silole-germole alternating copolymer 3 (FIG. 3), in which every other silicon or germanium atom in the polymer chain is also part of a silole or germole ring, was synthesized from the coupling of dichloro(tetraphenyl)germole and dilithio(tetraphenyl)silole. The latter is obtained in 39% yield from dichlorotetraphenylsilole by reduction with lithium, as illustrated in the equation of FIG. 3. The molecular weight of the silole-germole copolymer, M_{w}=5.5×10³, Mₙ=5.0×10³ determined by SEC (size exclusion chromatography) with polystyrene standards, is similar to that of polysiloles or polygermoles. All of the polymetalloles are extended oligomers with a degree of polymerization of about 10 to 16, rather than a true high M_{w} polymer; however, they can be cast into a thin film from solution and show polymer-like properties.

Illustrated in FIG. 4 are silole-silane alternating copolymers 4, 5, 6, 7, 8, which were also prepared from coupling of the silole dianion (Ph₄C₄Si)Li₂ with the corresponding silanes. Germole-silane alternation copolymers 9, 10, 11, 12 were also synthesized from the coupling of the germole dianion (Ph₄C₄Ge)Li₂ with the corresponding silanes, as illustrated in FIG. 4. These reactions generally employ reflux conditions in tetrahydrofuran under an argon atmosphere for about 72 hours. Some silole-silane copolymers have been synthesized previously and shown to be electroluminescent. Metallole-silane copolymers were developed so that they could be easily functionalized along the backbone by hydrosilation. The molecular weight of metallole-silane copolymers, M_{w}=4.1×10³˜6.2×10³, Mₙ=4.1×10³˜5.4×10³ determined by SEC, is similar to that of the polymetalloles.

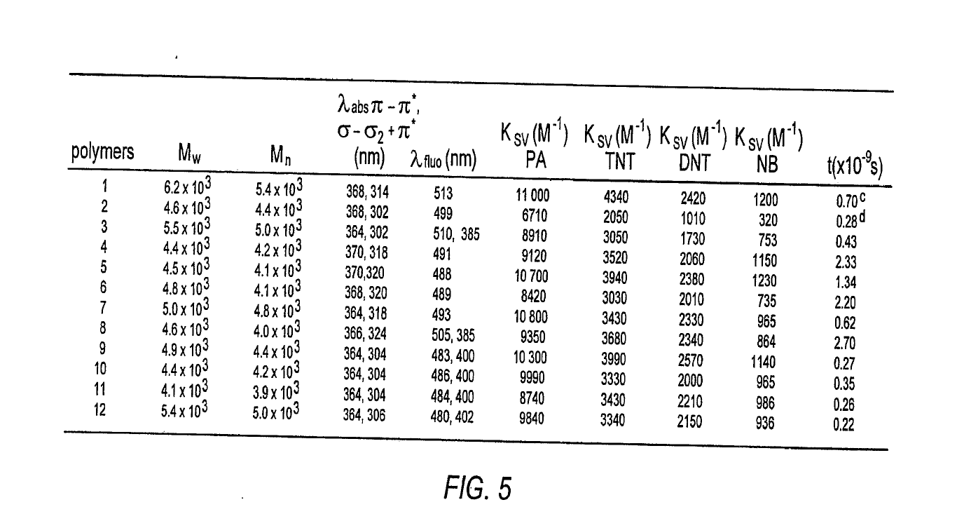

The molecular weights and polydisperity indices (PDI) of polymers 1-12 (FIG. 4) determined by gel permeation chromatography (GPC) are illustrated in Table 1 of FIG. 5.

Inorganic-organic poly(1,4-diethynylbenzene)metallole (DEB) type polymers may be obtained by hydrosilation of a dialkyne, specifically DEB, with a dihydrometallole using a catalyst such as chloroplatinic acid. FIGS. 21a-21c illustrate the reaction whereby the DEB type polymers are obtained according to embodiments of the invention. A reasonable extension of this principle includes hydrosilation and hydrogermylation of any organic diyne. A reasonable interpolation of this principle includes hydrosilation and hydrogermylation of organic dialkenes to obtain less conjugated polymers.

### Absorption and Fluorescence

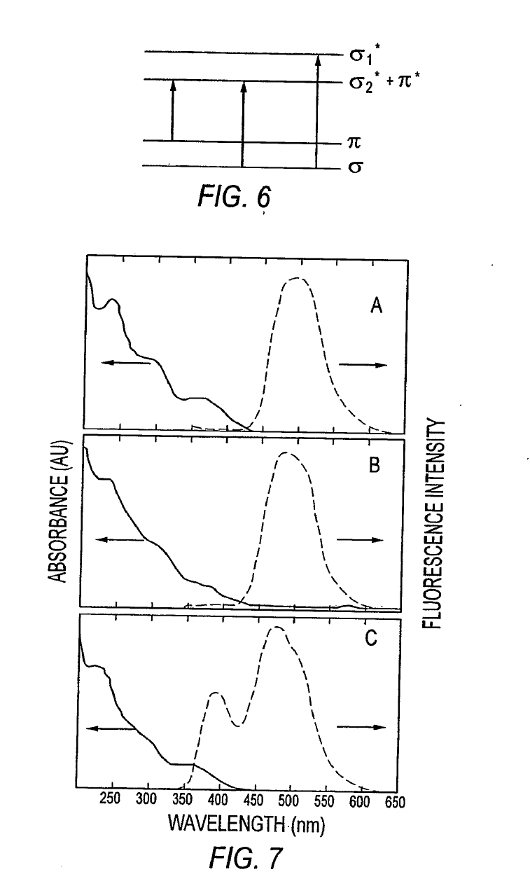

The UV-vis absorption and fluorescence spectral data for polymers 1-12 are also illustrated in Table 1 of FIG. 5. The poly(tetraphenyl)metalloles 1-3 and tetraphenylmetallole-silane copolymers 4-12 exhibit three absorption bands, which are ascribed to the π-π* transition in the metallole ring and the σ-(σ*+π*) and σ-σ* transitions in the M-M backbone. FIG. 6 illustrates a schematic energy-level diagram for polymetalloles and metallole-silane copolymers.

UV-vis absorption in THF (solid line) and fluorescence spectra in toluene (dotted line) for poly(tetraphenygermole) 2, silole-silane copolymer 4 and germole-silane copolymer 9 are shown in FIG. 7. Absorptions at a wavelength of about 370 nm for the poly(tetraphenylmetallole)s 1-3 and tetraphenylmetallole-silane copolymers 4-12 are ascribed to the metallole π-π* transition of the metallole moiety, which are about 89 to 95 nm red-shifted relative to that of oligo[1,1-(2,3,4,5-tetramethylsilole)] (λₘₐₓ=275 nm) and are about 75 to 81 nm red-shifted relative to that of oligo[1,1-(2,5-dimethyl-3,4-diphenylsilole)] (λₘₐₓ=289 nm). These red shifts are attributed to an increasing main chain length and partial conjugation of the phenyl groups to the silole ring.

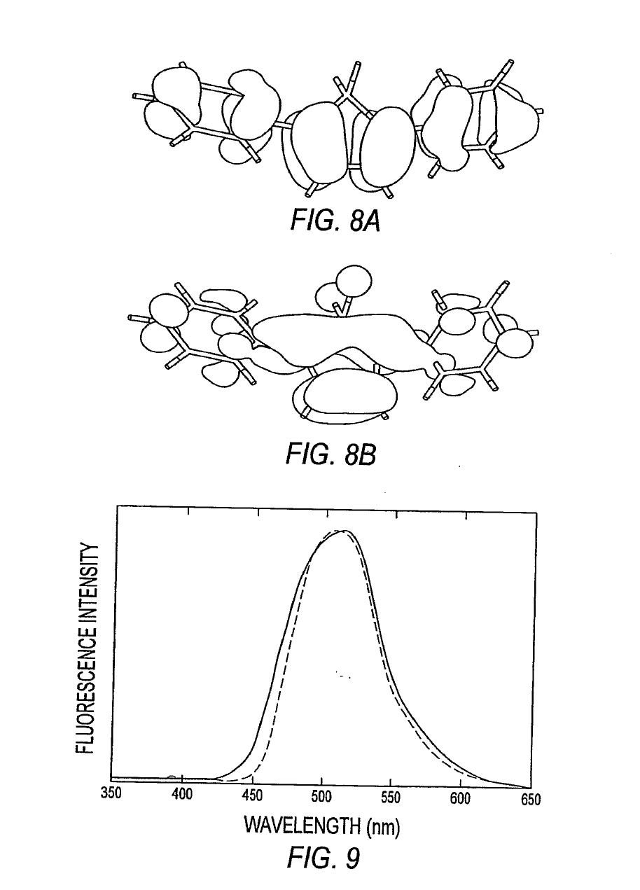

FIG. 8 shows the HOMO (A) and LUMO (B) of 2,5-diphenylsilole, Ph2C4SiH2, from the ab initio calculations at the HF/6-31G* level. Phenyl substituents at the 2,5 metallole ring positions may π-conjugate with the metallole ring LUMO. Second absorptions at wavelengths of 304 to 320 nm for the poly(tetraphenylmetallole)s 2-3 and tetraphenylmetallole-silane copolymers 4-12 are assigned to the σ-(σ₂*π*) transition, which parallels that of the poly(tetraphenyl)silole 1.

Polymetalloles 1-2 and silole-silane copolymers 4-7 exhibit one emission band (λₘₐₓ, 486 to 513 nm) when excited at 340 nm, whereas the others exhibit two emission bands with λₘₐₓ of 480-510 nm and 385-402 nm. The ratios of the two emission intensities are not concentration dependent, which indicates that the transition does not derive from an excimer. Emission peaks for germole-silane copolymers 9-12 are only 2 to 33 nm blue-shifted compared to the other polymers. FIG. 9 shows fluorescence spectra of the poly(tetraphenyl)silole in toluene solution (solid line) and in the solid state (dotted line). The bandwidth of the emission spectrum in solution is slightly larger than in the solid state. There is no shift in the maximum of the emission wavelength. This suggests that the polysilole exhibits neither π-stacking of polymer chains nor excimer formation.

The angles of C-M-C of dihydro(tetraphenyl)silole and dihydro(tetraphenyl)germole are 93.11° on C—Si—C and 89.76° on C—Ge—C, respectively. Polymerization might take place, since the tetraphenylmetalloles have small angles at C-M-C in the metallocyclopentadiene ring, which results in less steric hindrance at the metal center. In addition, the bulky phenyl groups of silole might prevent the formation of cyclic hexamer, which is often problematic in polysilane syntheses.

### Fluorescence Quenching With Nitroaromatic Analytes

A method of detection includes using a chemical sensor, namely a variety of luminescent copolymers having a metalloid-metalloid backbone such as Si—Si, Si—Ge, or Ge—Ge, or alternatively an inorganic-organic metallole-containing copolymer. While polymetalloles in various forms may be used to detect analytes, one embodiment includes casting a thin film of the copolymers to be employed in detecting the analyte, e.g., picric acid, DNT, TNT and nitrobenzene. Detection is achieved by measuring the quenching of the luminescence of the copolymer by the analyte. Accordingly, the present invention contemplates use of the polymetallole polymers and copolymers in any form susceptible to measurement of luminescence quenching. For example, since it is possible to measure fluorescence of solutions, other embodiments of the present method of detection may optionally include a polymetallole in solution phase, where powdered bulk polymer is dissolved in solution. Yet another embodiment includes producing a colloid of the polymer, which is a liquid solution with the polymer precipitated and suspended as nanoparticles.

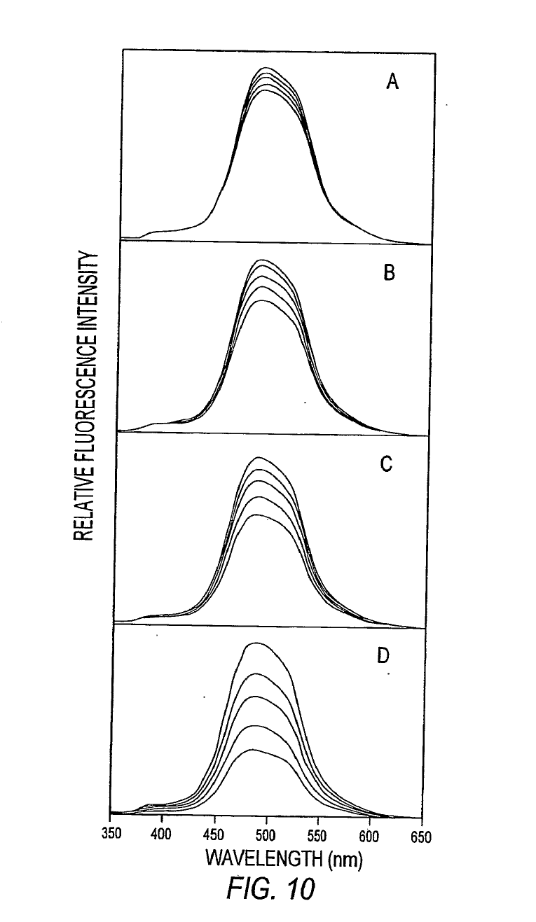

The detection method involves measurement of the quenching of luminescence of the polymetalloles 1-3 and metallole-silane copolymers 4-12 by the analyte, either visually or instrumentally (e.g., using a fluorescence spectrometer). For example, turning now to FIG. 10, when used to detect TNT, fluorescence spectra of a toluene solution of the metallole copolymers were obtained upon successive addition of aliquots of TNT. Photoluminescence quenching of the polymers 1-12 in toluene solutions were also measured with nitrobenzene, DNT, TNT and nitrobenzene. The relative efficiency of photoluminescence quenching of metallole copolymers is unique for TNT, DNT, and nitrobenzene, respectively, as indicated in FIG. 10 by the values of K determined from the slopes of the steady-state Stern-Volmer plots. FIG. 10 demonstrates that each copolymer has a unique ratio of quenching efficiency to the corresponding analyte.

Certain impurities of TNT may contribute to improved results. It was synthesized by nitration of dinitrotoluene and recrystallized twice from methanol. A third recrystallization produces the same results as the twice-recrystallized material. When the quenching experiment was undertaken without recrystallization of TNT, higher (ca. 10×) quenching percentages are obtained. Presumably, impurities with higher quenching efficiencies are present in crude TNT.

The Stern-Volmer equation, which is (I_{O}/I)−1=K_{SV}[A], is used to quantify the differences in quenching efficiency for various analytes. In this equation, I_{O} is the initial fluorescence intensity without analyte, and I is the fluorescence intensity with added analyte of concentration [A], and K_{SV} is the Stem-Volmer constant.

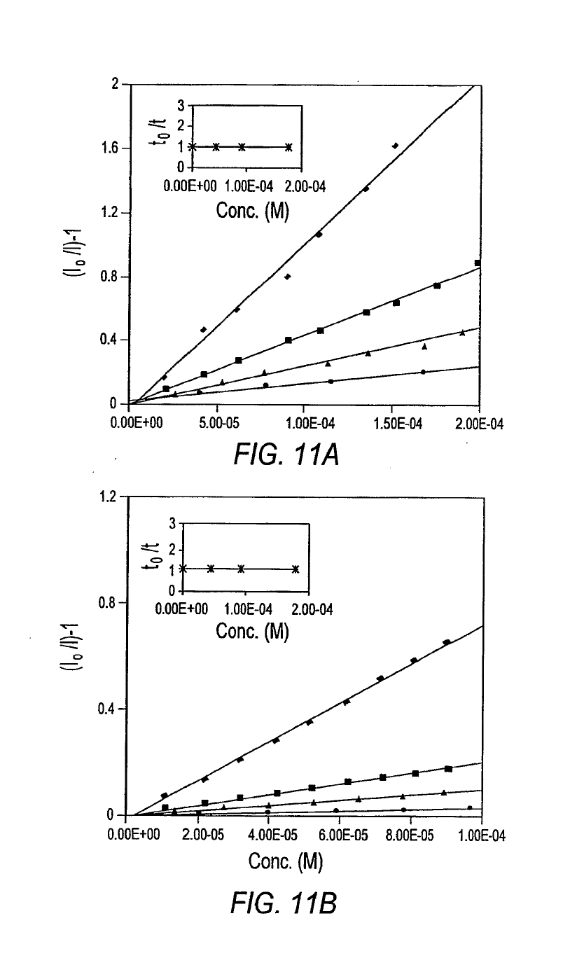

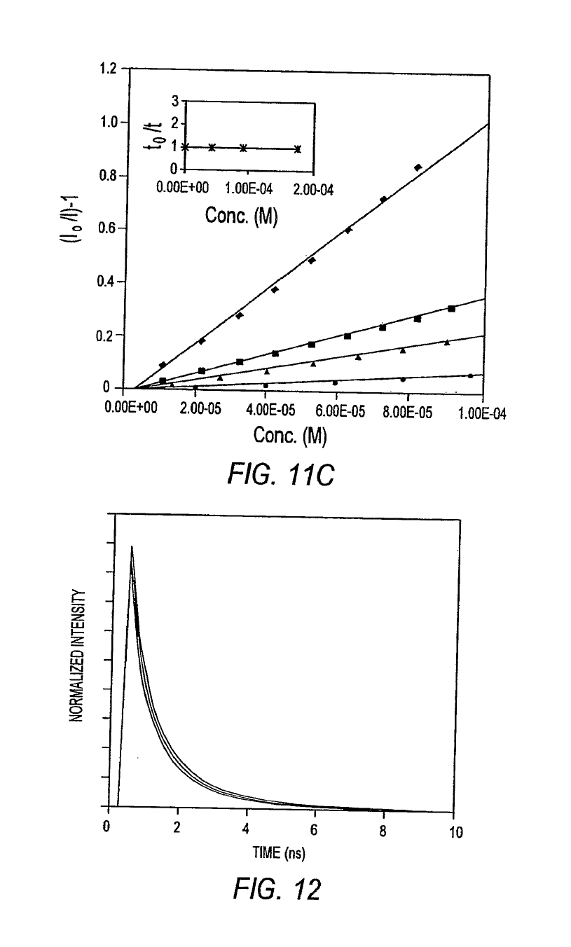

FIG. 11 shows the Stem-Volmer plots of polysilole 1, polygermole 2, and silole-silane copolymer 8 for each analyte. A linear Stern-Volmer relationship was observed in all cases, but the Stem-Volmer plot for picric acid exhibits an exponential dependence when its concentration is higher than 1.0×10⁻⁴ M. A linear Stern-Volmer relationship may be observed if either static or dynamic quenching process is dominant. Thus, in the case of higher concentrations of picric acid, the two processes may be competitive, which results in a nonlinear Stem-Volmer relationship. This could also arise from aggregation of analyte with chromophore.

Photoluminescence may arise from either a static process, by the quenching of a bound complex, or a dynamic process, by collisionally quenching the excited state. For the former case, K_{SV} is an association constant due to the analyte-preassociated receptor sites. Thus, the collision rate of the analyte is not involved in static quenching and the fluorescence lifetime is invariant with the concentration of analyte. With dynamic quenching, the fluorescence lifetime should diminish as quencher is added.

A single “mean” characteristic lifetime (τ) for polymetalloles and metallole-silane copolymers 1-12 has been measured and summarized in Table 1 of FIG. 5. Luminescence decays were not single-exponential in all cases. Three lifetimes were needed to provide an acceptable fit over the first few nanoseconds. The amplitudes of the three components were of comparable importance (the solvent blank made no contribution). These features suggest that the complete description of the fluorescence is actually a continuous distribution of decay rates from a heterogeneous collection of chromophore sites. Because the oligomers span a size distribution, this behavior is not surprising. The mean lifetime parameter reported is an average of the three lifetimes determined by the fitting procedure, weighted by their relative amplitudes. This is the appropriate average for comparison with the “amount” of light emitted by different samples under different quenching conditions, as has been treated in the literature. Given this heterogeneity, possible long-lived luminescence that might be particularly vulnerable to quenching has been a concern. However, measurements with a separate nanosecond laser system confirmed that there were no longer-lived processes other than those captured by the time-correlated photon counting measurement and incorporated into Table 1 of FIG. 5.

It is notable that polysilole 1 and silole-silane copolymers 4-8 have about 3 to 11 times longer fluorescence lifetimes than polygermole 2 and germole-silane copolymers 9-12. Fluorescence lifetimes in the thin films (solid state) for polysilole 1 and polygermole 2 are 2.5 and 4.2 times longer than in toluene solution, respectively. The fluorescence lifetimes as a function of TNT concentration were also measured and are shown in the inset of FIG. 11 for polymers 1, 2, and 8. No change of mean lifetime was observed by adding TNT, indicating that the static quenching process is dominant for polymetalloles and metallole-silane copolymers 1-12 (FIG. 12). Some issues with such analyses have been discussed in the literature. This result suggests that the polymetallole might act as a receptor and a TNT molecule would intercalate between phenyl substituents of the metallole moieties (FIG. 1).

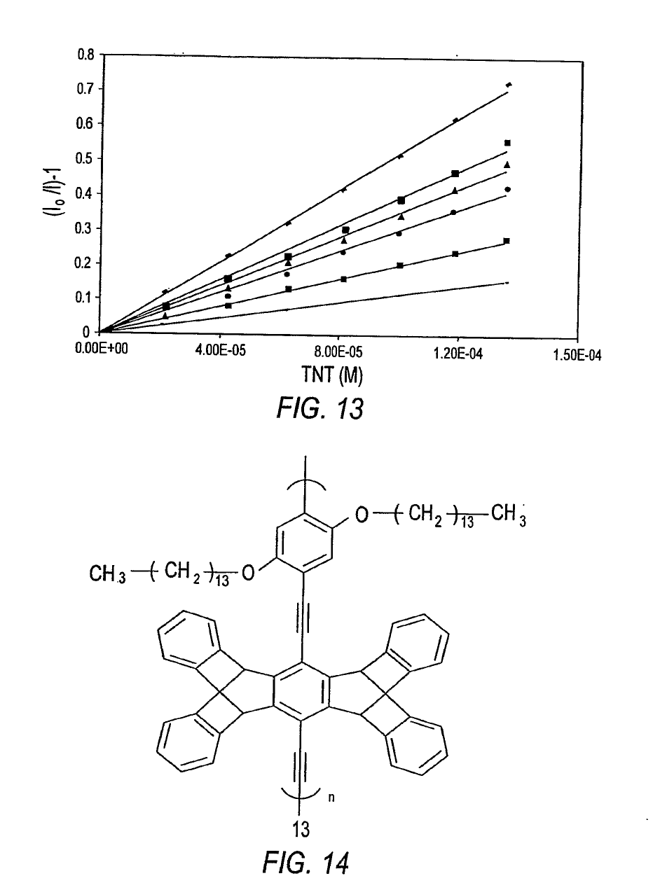

For chemosensor applications, it is useful to have sensors with varied responses. Each of the 12 polymers exhibits a different ratio of the photoluminescence quenching for picric acid, TNT, DNT, and nitrobenzene and a different response with the same analyte. The use of sensor arrays is inspired by the performance of the olfactory system to specify an analyte. FIG. 13 displays the Stern-Volmer plots of polymers 1, 2, 4, 5, and 6 for TNT, indicating that the range of photoluminescence quenching efficiency for TNT is between 2.05×10³ and 4.34×10³ M⁻¹. The relative efficiencies of photoluminescence quenching of poly(tetraphenylmetallole)s 1-3 and tetraphenyl-metallole-silane copolymers 4-12 were obtained for picric acid, TNT, DNT, and nitrobenzene, as indicated by the values of Ksv determined from the slopes of the steady-state Stern-Volmer plots and summarized in Table 1 of FIG. 5. Polymer 13, which is illustrated in FIG. 14, is an organic pentiptycene-derived polymer for comparison. The metallole copolymers are more sensitive to TNT than the organic pentiptycene-derived polymers in toluene solution. For example, polysilole 1 (4.34×10³ M⁻¹) has about a 370% better quenching efficiency with TNT than organic pentiptycene-derived polymer (1.17×10³ M⁻¹).

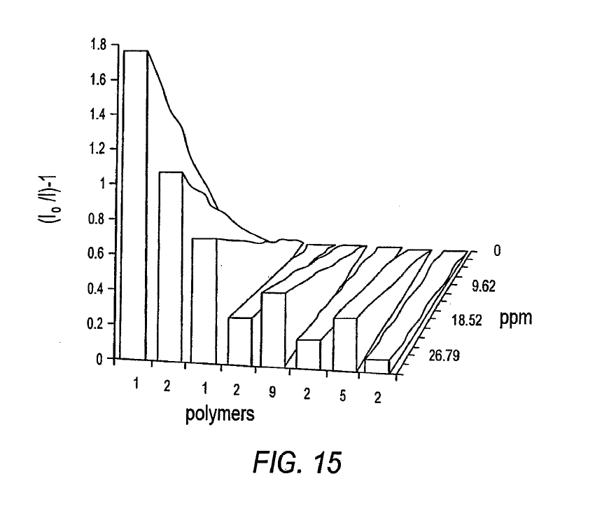

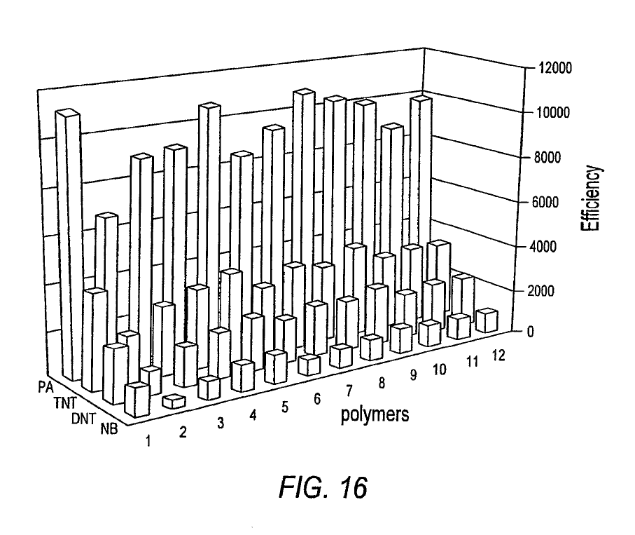

The trend in Stem-Volmer constants usually reflects an enhanced charge-transfer interaction from metallole polymer to analyte. For example, the relative efficiency of photoluminescence quenching of polysilole 1 is about 9.2:3.6:2.0:1.0 for picric acid, TNT, DNT, and nitrobenzene, respectively. Although polysilole 1 shows best photoluminescence quenching efficiency for picric acid and TNT, polymer 9 and 5 exhibit best quenching efficiency for DNT and nitrobenzene, respectively. (FIG. 15) Polygermole 2 has the lowest quenching efficiency for all analytes. Since the polymers 1-12 have similar molecular weights, the range of quenching efficiencies with the same analyte would be expected to be small. Polysilole 1 (11.0×10³M⁻¹ and 4.34×10³ M⁻¹) exhibits 164% and 212% better quenching efficiency than polygermole 2 (6.71×10³ and 2.05×10³ M⁻¹) with picric acid and TNT, respectively. Polymer 9 (2.57×10³ M⁻¹) has 253% better quenching efficiency than polymer 2 (1.01×10³ M⁻¹) with DNT. Polymer 5 (1.23×10³ M⁻¹) has 385% better quenching efficiency than metallole polymer 2 (0.32×10³ M⁻¹) with nitrobenzene. FIG. 16 illustrates how an analyte might be specified using an array of multi-sensors.

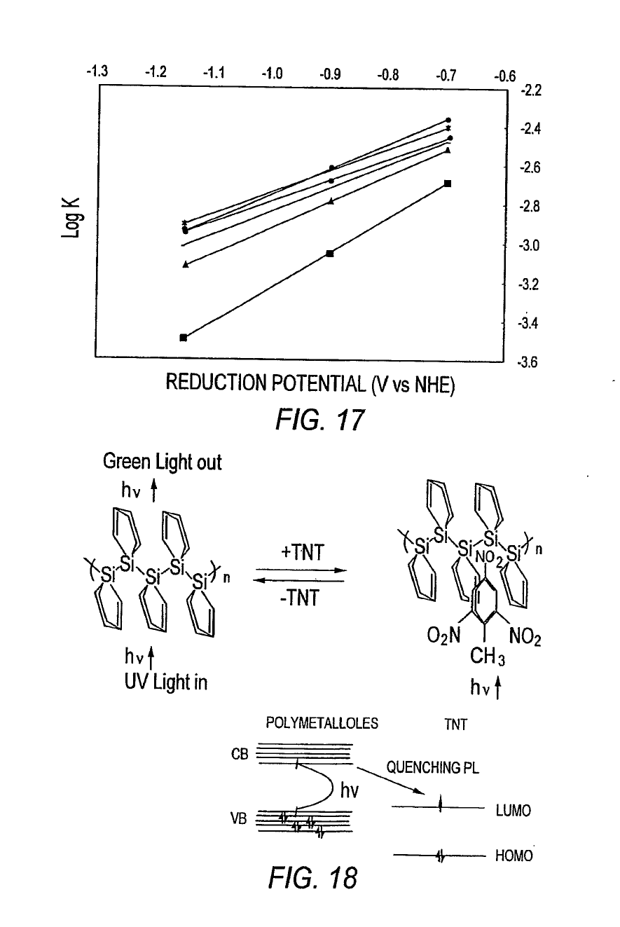

FIG. 17 shows a plot of log Ksv vs. reduction potential of analytes. All metallole polymers exhibit a linear relationship, even though they have different ratios of photoluminescence quenching efficiency to analytes. This result indicates that the mechanism of photoluminescence quenching is primarily attributable to electron transfer from the excited metallole polymers to the LUMO of the analyte. Because the reduction potential of TNT (−0.7 V vs NUE) is less negative than that of either DNT (−0.9 V vs NHE) or nitrobenzene (−1.15 V vs NHE), it is detected with highest sensitivity. A schematic diagram of the electron-transfer mechanism for the quenching of photoluminescence of the metallole polymers with analyte is shown in FIG. 18. Optical excitation produces an electron-hole pair, which is delocalized through the metallole copolymers. When an electron deficient molecule, such as TNT is present, electron-transfer quenching occurs from the excited metallole copolymer to the LUMO of the analyte. The observed dependence of Ksv on analyte reduction potential suggests that for the static quenching mechanism, the polymer-quencher complex luminescence intensity depends on the electron acceptor ability of the quencher. An alternative explanation would be that the formation constant (Ksv) of the polymer-quencher complex is dominated by a charge-transfer interaction between polymer and quencher and that the formation constant increases with quencher electron acceptor ability.

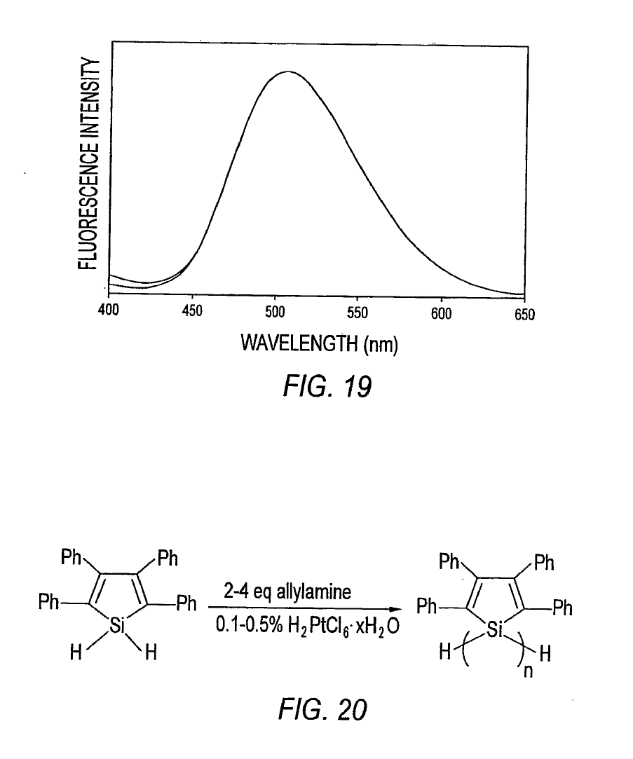

An important aspect of the metallole copolymers is their relative insensitivity to common interferents. Control experiments using both solutions and thin films of metallole copolymers (deposited on glass substrates) with air displayed no change in the photoluminescence spectrum. Similarly, exposure of metallole copolymers both as solutions and thin films to organic solvents such as toluene, THF, and methanol or the aqueous inorganic acids H₂SO₄ and HF produced no significant decrease in photoluminescence intensity. FIG. 19 shows that the photoluminescence spectra of polysilole 1 in toluene solution display no quenching of fluorescence with 4 parts per hundred of THF. The ratio of quenching efficiency of polysilole 1 with TNT vs benzoquinone is much greater than that of polymer 13. The Ksv value of 4.34×10³ M⁻¹ of polysilole 1 for TNT is 640% greater than that for benzoquinone (Ksv=674 M⁻¹). The organic polymer 13, however, only exhibits a slightly better quenching efficiency for TNT (Ksv=1.17×10³ M⁻¹) (ca. 120%) compared to that (Ksv=998 M⁻¹) for benzoquinone. This result indicates that polysilole 1 exhibits less response to interferences and greater response to nitroaromatic compounds compared to the pentiptycene-derived polymer 13.

Statistical Estimates of Detection Limit from Extrapolation of Stem-Volmer Quenching Data:

An extrapolated detection limit of ˜1.5 ppt for instant detection with a fluorescence spectrometer at the 95% confidence limit is estimated using the Stern Volmer Equation: log(I₀/I)−1 vs [TNT] in ppb. Of course, this is for solution data and with a spectrometer, which is not optimized for detection at a single wavelength.

### Preparation of Nitroaromatic-Explosives Detecting Metallole-Containing Polymers

All synthetic manipulations were carried out under an atmosphere of dry dinitrogen gas using standard vacuum-line Schlenk techniques. All solvents were degassed and purified prior to use according to standard literature methods: diethyl ether, hexanes, tetrahydrofuran, and toluene purchased from Aldrich Chemical Co. Inc. were distilled from sodium/benzophenone ketal. Spectroscopic grade of toluene from Fisher Scientific was used for the fluorescent measurement. NMR grade deuteriochloroform was stored over 4 Å molecular sieves. All other reagents (Aldrich, Gelest) were used as received or distilled prior to use. NMR data were collected with Varian Unity 300, 400, or 500 MHz spectrometers (300.1 MHz for ¹H NMR, 75.5 MHz for ¹³C NMR and 99.2 MHz for ²⁹Si NMR) and all NMR chemical shifts are reported in parts per million (δ ppm); downfield shifts are reported as positive values from tetramethylsilane (TMS) as standard at 0.00 ppm. The ¹H and ¹³C chemical shifts are reported relative to CHCl₃ (δ 77.0 ppm) as an internal standard, and the ²⁹Si chemical shifts are reported relative to an external TMS standard.

NMR spectra were recorded using samples dissolved in CDCl₃, unless otherwise stated, on the following instrumentation. ¹³C NMR were recorded as proton decoupled spectra, and ²⁹Si NMR were recorded using an inverse gate pulse sequence with a relaxation delay of 30 seconds. The molecular weight was measured by gel permeation chromatography using a Waters Associates Model 6000A liquid chromatograph equipped with three American Polymer Standards Corp. Ultrastyragel columns in series with porosity indices of 10³, 10⁴, and 10⁵ Å, using freshly distilled THF as eluent.

The polymer was detected with a Waters Model 440 ultraviolet absorbance detector at a wavelength of 254 nm, and the data were manipulated using a Waters Model 745 data module. Molecular weight was determined relative to calibration from polystyrene standards. Fluorescence emission and excitation spectra were recorded on a Perkin-Elmer Luminescence Spectrometer LS 50B. Monomers, 1,1-dichloro-2,3,4,5-tetraphenylsilole, 1,1-dichloro-2,3,4,5-tetraphenylgermole, 1,1-dilithio-2,3,4,5-tetraphenylsilole, and 1,1-dilithio-2,3,4,5-tetraphenylgermole were synthesized by following the procedures described in the literature. All reactions were performed under Ar atmosphere.

Polymetalloles 1, 2, and 3 were synthesized by following the procedures described in the literature.

Preparation of silole-silane copolymers, (silole-SiR¹R²)ₙ:

Stirring of 1,1-dichloro-2,3,4,5-tetraphenylsilole (5.0 g, 11.0 mmol) with lithium (0.9 g, 129.7 mmol) in THF (120 mL) for 8 h at room temperature gave a dark yellow solution of silole dianion. After removal of excess lithium, 1 mol equiv of corresponding silanes, R¹R²SiCl₂ (11.0 mmol) was added slowly to a solution of tetraphenylsilole dianion, and stirred at room temperature for 2 hours. The resulting mixture was refluxed for 3 days. The reaction mixture was cooled to room temperature and quenched with methanol. Then the volatiles were removed under reduced pressure. THF (20 mL) was added to the residue and polymer was precipitated by slow addition of the solution into 700 mL of methanol. The third cycle of dissolving-precipitation followed by freeze-drying gave the polymer as yellow powder.

For (silole)ₙ(SiMeH)ₘ(SiPhH)ₒ, each 5.5 mmol of SiMeHCl₂ and SiPhHCl₂ were slowly added into a THF solution of silole dianion. In case of (silole-SiH₂)ₘ, after addition of the xylene solution of SiH₂Cl₂ (11.0 mmol), the resulting mixture was stirred for 3 days at room temperature instead of refluxing.

Selected data for (silole-SiMeH)ₙ, 4; Yield=2.10 g (44.5%); ¹H-NMR (300.134 MHz, CDCl₃): δ=−0.88-0.60 (br. 3H, Me), 3.06-4.89 (br. 1H, SiH), 6.16-7.45 (br. 20H, Ph); ¹³C{H} NMR (75.469 MHz, CDCl₃): δ=0.61-1.69 (br. Me), 123.87-131.75, 137.84-145.42, 153.07-156.73 (br. m, Ph); ²⁹Si NMR (71.548 MHz, inversed gated decoupling, CDCl₃): δ=−29.22 (br. silole), −66.61 (br. SiMeH). GPC: Mw=4400, Mw/Mn=1.04. Fluorescence (conc.=10 mg/L); λₑₘ=492 nm at λₑₓ=340 nm.

Selected data for (silole-SiPhH)ₙ, 5; Yield=2.00 g (37.0%); ¹H NMR (300.134 MHz, CDCl₃): δ=3.00-4.00 (br. 1H, SiH), 6.02-7.97 (br. 20H, Ph); ¹³C{H} NMR (75.469 MHz, CDCl₃): δ=123.64-143.98, 152.60-157.59 (br. m, Ph); ²⁹Si NMR (71.548 MHz, inversed gated decoupling, CDCl₃): δ=−37.51 (br. silole), −71.61 (br. SiPhH). GPC: Mw=4500, Mw/Mn=1.09, determined by SEC with polystyrene standards; Fluorescence (conc.=10 mg/L); λₑₘ=487 nm at λₑₓ=340 nm.

Selected data for (silole)ₙ(SiMeH)_{0.5n}(SiPhH)_{0.5n}, 6; Yield=2.10 g (41.5%); ¹H NMR (300.134 MHz, CDCl₃): δ=−0.67-0.40 (br. 3H, Me), 3.08-4.98 (br. 2H, SiH), 6.00-7.82 (br. 55H, Ph); ¹³C{H} NMR (75.469 MHz, CDCl₃): δ=−0.85-1.76 (br. Me), 122.06-147.25, 153.11-157.26 (br. m, Ph); ²⁹Si NMR (71.548 MHz, inversed gated decoupling, CDCl₃): δ=−28.61 (br. silole), −59.88 (br. SiMeH and SiPhH). GPC: Mw=4800, Mw/Mn=1.16, determined by SEC with polystyrene standards; Fluorescence (conc.=10 mg/L); λₑₘ=490 nm at λₑₓ=340 nm.

Selected data for (silole-SiH₂)ₙ, 8; Yield=2.05 g (44.9%); ¹H NMR (300.134 MHz, CDCl₃): δ=3.00-4.96 (br. 2H, SiH₂), 6.12-7.72 (br. 20H, Ph); ¹³C{H} NMR (75.469 MHz, CDCl₃): δ=122.08-132.78, 136.92-146.25, 152.81-160.07 (br. m, Ph); ²⁹Si NMR (71.548 MHz, inversed gated decoupling, CDCl₃): δ=−30.95 (br. silole), −51.33 (br. SiH₂). ratio of n:m=1.00:0.80; GPC: Mw=4600, Mw/Mn=1.14, determined by SEC with polystyrene standards; Fluorescence (conc.=10 mg/L); λₑₘ=499 nm at λₑₓ=340 nm.

Selected data for (silole-SiPh₂)ₙ, 7; Yield=2.93 g (47.0%); ¹H NMR (300.134 MHz, CDCl₃): δ=6.14-7.82 (br. 20H, Ph); ¹³C{H} NMR (75.469 MHz, CDCl₃): δ=122.08-146.25 (br. m, Ph), 152.81-160.07 (silole ring); GPC: Mw=5248, Mw/Mn=1.05, determined by SEC with polystyrene standards; Fluorescence (conc.=10 mg/L); λₑₘ=492 nm at λₑₓ=340 nm.

### Preparation of Germole-Silane Copolymers, (germole-SiR¹R²)ₙ:

The procedure for synthesizing all germole-silane copolymers was similar to that for silole-silane copolymers. For (germole)ₙ(SiMeH)_{0.5n}(SiPhM_{0.5n}, each 5.0 mmol of SiMeHCl₂ and SiPhHCl₂ were added slowly into a THF solution of germole dianion. The resulting mixture was stirred for 3 days at room temperature.

Selected data for (germole-SiMeH)ₙ, 9; Yield=2.03 g (43%); ¹H NMR (300.134 MHz, CDCl₃): δ=−0.21-0.45 (br. 2.4H, Me), 5.14-5.40 (br. 0.8H, SiH), 6.53-7.54 (br. 20H, Ph); ¹³C{H} NMR (75.469 MHz, CDCl₃): δ=−9.70-−8.15 (br. Me), 125.29-130.94, 139.08-148.12, 151.29-152.88 (br. m, Ph); ²⁹Si NMR (71.548 MHz, inversed gated decoupling, CDCl₃): δ=−50.40 (br. SiMeH); GPC: Mw=4900, Mw/Mn=1.12, determined by SEC with polystyrene standards; UV (conc.=10 mg/L); δ_{abs}=296, 368 nm; Fluorescence (conc.=10 mg/L); λₑₘ=401, 481 nm at λₑₓ=340 nm.

Selected data for (germole-SiPhH)ₙ, 10; Yield=2.13 g (40%); ¹H NMR (300.134 MHz, CDCl₃): δ=4.71 (br. 1.0H, SiH), 6.30-7.60 (br. 25H, Ph); ¹³C{H} NMR (75.469 MHz, CDCl₃): δ=125.50-144.50, 151.50-153.00 (br. m, Ph); ²⁹Si NMR (71.548 MHz, inversed gated decoupling, CDCl₃): δ=−56.81 (br. SiPhH); GPC: Mw=4400, Mw/Mn=1.06, determined by SEC with polystyrene standards; UV (conc.=10 mg/L); λ_{abs}=294, 362 nm; Fluorescence (conc.=10 mg/L); λₑₘ=401, 486 nm at λₑₓ=340 nm.

Selected data for (germole)ₙ(SiMeH)_{0.5n}(SiPhH)_{0.5n}, 11; Yield=2.01 g (40%); ¹H NMR (300.134 MHz, CDCl₃): δ=−0.04-0.42 (br. 3H, 4.94 (br. 2H, SiH), 6.33-7.66 (br. 25H, Ph); ¹³C{H} NMR (75.469 MHz, CDCl₃): δ=124.31-130.66, 138.43-152.54 (br. m, Ph); ²⁹Si NMR (71.548 MHz, inversed gated decoupling, CDCl₃): δ=−63.01 (br. SiMeH and SiPhH): 0.71; GPC: Mw=4100, Mw/Mn=1.06, determined by SEC with polystyrene standards; UV (conc.=10 mg/L); λ_{abs}=290, 364 nm; Fluorescence (conc.=10 mg/L); λₑₘ=399, 483 nm at λₑₓ=340 nm.

Selected data for (germole-SiPh₂)ₙ, 12; Yield=3.23 g (48%); ¹H NMR (300.134 MHz, CDCl₃): δ=6.21-7.68 (br. 30H, Ph); ¹³C{H} NMR (75.469 MHz, CDCl₃): δ=125.15-141.40 (br. m, Ph), 151.12-153.99 (germole ring carbon); GPC: Mw=5377, Mw/Mn=1.09, determined by SEC with polystyrene standards; UV (conc.=10 mg/L); λ_{abs}=298, 366 nm; Fluorescence (conc.=10 mg/L); λₑₘ=400, 480 nm at λₑₓ=340 nm.

Preparations for other metallole-silane and metallole-germane copolymers such as tetraalkylmetallole-silane copolymers and tetraarylmetallole-germane copolymers can be prepared by the above method described.

Preparation of Poly(tetraphenyl)silole and Poly(tetraphenyl)germole by Catalytic Dehydrocoupling—Preparation of polymetallole: 1,1-dihydro-2,3,4,5-tetraphenylsilole or germole were prepared from the reduction of 1,1-dichloro-2,3,4,5-tetraphenylsilole or germole with 1 mol equiv of LiAlH₄. Additionally, an alternate method to prepare the dihydrometallole is to add dichlorosilane (25% in xylenes) to an solution of tetraphenylbutadiene dianion in ether, as described in the literature. Reaction conditions for preparing the polygermole are the same as those for polysilole. 1,1-dihydro-2,3,4,5-tetraphenylsilole (1.0 g, 2.59 mmol) and 1-5 mol % of RhCl(PPh₃)₃ or Pd(PPh₃)₄ in toluene (10 mL) were placed under an Ar atmosphere and degassed through 3 freeze-pump-thaw cycles. The reaction mixture was vigorously refluxed for 72 h. The solution was passed rapidly through a Florisil column and evaporated to dryness under Ar atmosphere. 1 mL of THF was added to the reaction mixture and the resulting solution was then poured into 10 mL of methanol. Poly(tetraphenyl)silole, 1, was obtained as a pale yellow powder after the third cycle of dissolving-precipitation followed by freeze-drying. An alternative method for poly(tetraphenyl)silole preparation is as follows. 1,1-dihydro-2,3,4,5-tetraphenylsilole (1.0 g, 2.59 mmol) and 0.1-0.5 mol % H₂PtCl₆.xH₂O and 2-5 mol equivalents of allylamine in toluene (10 mL) were vigorously refluxed for 24 hours. The solution was passed through a sintered glass frit and evaporated to dryness under an Ar atmosphere. Three dissolving-precipitation cycles with THF and methanol were performed as stated above to obtain 1. The molecular weights of polymers were obtained by GPC. 1,1-dihydro-2,3,4,5-tetraphenylsilole with RhCl(PPh₃)₃, 1: isolated yield=0.81 g, 82%, M_{w}=4355, M_{w}/Mₙ=1.02, determined by SEC with polystyrene standards; 1,1-dihydro-2,3,4,5-tetraphenylsilole with Pd(PPh₃)₄, 1: 0.84 g, 85%, M_{w}=5638, M_{w}/Mₙ=1.10). 1,1-dihydro-2,3,4,5-tetraphenylgermole with RhCl(PPh₃)₃, poly(tetraphenyl)germole: 0.80 g, 81%, M_{w}=3936, M_{w}/Mₙ=1.01; 1,1-dihydro-2,3,4,5-tetraphenylgermole with Pd(PPh₃)₄, poly(tetraphenyl)germole: 0.81 g, 82%, M_{w}=4221, M_{w}/Mₙ=1.02) ¹H NMR (300.133 MHz, CDCl₃): δ=6.30-7.90 (br, m, Ph); ¹³C{H} NMR (75.403 MHz, CDCl₃ (δ=77.00)): δ=124-130 (br, m, Ph), 131-139 (germole carbons). If less vigorous reflux conditions are used, with the RhCl(PPh₃)₃ and Pd(PPh₃)₄ catalysts, then corresponding dimers form along with lesser amounts of polymer. The dimer is less soluble and crystallizes from toluene.

### Preparation of Poly(1,4-diethynylbenzene)2,3,4,5-tetraphenylsilole (PDEBsilole)

1,1 dihydro-2,3,4,5-tetraphenylsilole (250 mg, 0.65 mmol), 1,4-diethynylbenzene (100 mg, 0.80 mmol), and 0.1-0.5 mol % H₂PtCl₆.xH₂O were vigorously refluxed in toluene (10 mL), under argon for 4 hours. The dark orange solution was passed through a sintered glass frit and evaporated to dryness. The remaining solid was dissolved in 1 ml of THF, precipitated with 10 ml of methanol, and collected by filtration on a sintered glass frit. The precipitation was repeated twice more and the polymer was obtained as a yellow solid (0.17 g, 51%). The molecular weight of the polymer was determined by GPC with polystyrene standards. M_{w}=6,198, M_{w}/Mₙ=1.822; ¹H NMR (300.075 MHz, CDCl₃): δ 6.60-7.20 (br, 24H, silole Ph, ═CH—Si, and ═CH—Ph), δ 7.40 (br, 4H, phenylene Ph); UV (conc.=20 mg/L); λ_{abs}=302, 378 nm; Fluorescence (conc. 20 mg/L); λₑₘ=500 nm (λₑₓ=360 nm).

### Preparation of Poly(1,4-diethynylbenzene)2,3,4,5-tetraphenylgermole (PDEBgermole)

1,1-dihydro-2,3,4,5-tetraphenylgermole (100 mg, 0.23 mmol), 1,4-diethynylbenzene (34 mg, 0.26 mmol), and 0.1-0.5 mol % H₂PtCl₆.xH₂O were vigorously refluxed in toluene (10 mL), under argon for 12 hours. The catalyst was removed by filtration, and the filtrate then evaporated to dryness. The remaining solid was dissolved in THF (1 mL) and precipitated by subsequent addition of methanol (10 mL). The polymer was collected by filtration and dried to afford the yellow powder (0.095 g, 73%). Molecular weights determined by GPC: M_{w}=4800, M_{w}/Mₙ=1.6; ¹H NMR (300.075 MHz, CDCl₃): δ 6.50-7.60 (br, silole Ph, ═CH—Ge, and ═CH—Ph, phenylene H); UV-Vis (Toluene): λ_{abs}=290, 362 nm; Fluorescence (Toluene): λₑₘ=475 nm (λₑₓ=360 nm).

### Preparation of Poly(1,4-diethynylbenzene)silafluorene (PDEBSF)

1,1 dihydrosilafluorene (0.25 g, 1.37 mmol), 1,4-diethynylbenzene (0.19 g, 1.51 mmol), and 0.1-0.5 mol % H₂PtCl₆.xH₂O were vigorously refluxed in toluene (3 mL), under argon for 24 hours. The dark orange/red solution was filtered and evaporated to dryness. The remaining solid was dissolved in 4 ml of THF, precipitated with 40 ml of methanol. The white solid (0.17 g, 34%) was collected by filtration on a sintered glass frit. The molecular weight of the polymer was determined by GPC with polystyrene standards. M_{w}=1,957, M_{w}/Mₙ=1.361; ¹H NMR (300.075 MHz, CDCl₃): δ 6.00-8.00 (br, 16H, silafluorene H-Ph, ═CH—Si, and ═CH-Ph); UV (conc.=20 mg/L); λ_{abs}=292 nm; Fluorescence (conc. 0.2 mg/L); λₑₘ=341, 353 nm at λₑₓ=292 nm.

### Preparation and Characterization of Polysilafluorene (PSF)

The high energy of the excited state in the UV luminescent polysilafluorene offers an increased driving force for electron transfer to the explosive analyte and improved detection limits by electron transfer quenching, which should be applicable for any UV emitting conjugated organic or inorganic polymer.

1,1-dihydrosilafluorene (500 mg, 2.7 mmol) and 0.5 mol % H₂PtCl₆.xH₂O were stirred in toluene (3 mL) at 80° C. under argon for 24 hours. The orange-brown solution was filtered while warm and evaporated to dryness. The remaining solid was dissolved in 3 mL of THF and precipitated with the addition of 30 mL of methanol. The resulting light orange-white solid was collected by vacuum filtration (0.101 g, 20%). The molecular weight of the polymer was determined by GPC with polystyrene standards. M_{w}=576, M_{w}/Mₙ=1.074; ¹H NMR (300.075 MHz, CDCl₃): δ 6.60-7.90 (br, 8H, silafluorene H-Ph), δ 4.62 (weak s, terminal Si—H); UV (conc.=20 mg/L); λ_{abs}=392 nm; Fluorescence (conc. 0.2 mg/L); λₑₘ=342, 354 nm, at λₑₓ=292 nm.

Detection limits of trinitrotoluene (TNT), dinitrotoluene (DNT), picric acid (PA), 2,2′-dimethyl-2,2′-dinitrobutane (DMNB), orthomononitrotoluene (OMNT), and paramononitrotoluene (PMNT) were detemined by fluorescence quenching of polysilole, polyDEBsilole, polygermole, polyDEBgermole, PSF, polyDEBSF, and ExPray. (DEB=diethynylbenzene.) The emission of PSF is centered in the UV, so detection limits with a UV camera are expected to be even better than those determined visually.

### Preparation and Characterization of Polygermafluorene (PGF)

1,1-dihydrogermafluorene (0.1 g, 0.44 mmol) and 0.5 mol % H₂PtCl₆.xH₂O were refluxed in toluene (4 mL) under argon for 24 hours. The thick orange solution was filtered while warm and evaporated to dryness. The remaining solid was dissolved in 2 mL of THF and precipitated with 22 mL of methanol. The resulting light orange-white solid was collected by vacuum filtration (0.010 g, 10%). The molecular weight of the polymer was determined by GPC with polystyrene standards. M_{w}=890, M_{w}/Mₙ=1.068; ¹H NMR (300.075 MHz, CDCl₃): δ 6.40-7.90 (br, 8H, silafluorene H-Ph).

### Preparation and Characterization of Poly(1,4-diethynylbenzene)germafluorene (PDEBGF)

1,1 dihydrogermafluorene (0.15 g, 0.66 mmol), 1,4-diethynylbenzene (0.092 g, 0.73 mmol), and 0.1-0.5 mol % H₂PtCl₆.xH₂O were vigorously refluxed in toluene (4 mL), under argon for 24 hours. The dark orange-red solution was filtered and evaporated to dryness. The remaining solid was dissolved in 4 ml of THF and precipitated with 40 ml of methanol. The light orange solid (0.021 g, 15%) was collected by filtration on a sintered glass frit. The molecular weight of the polymer was determined by GPC with polystyrene standards. M_{w}=1,719, M_{w}/Mₙ=1.872; ¹H NMR (300.075 MHz, CDCl₃): δ 6.00-8.00 (br, 16H, germafluorene H-Ph, ═CH—Si, and ═CH-Ph).

### Experimental Results and Data for Nitroaromatics

The method of explosives detection is through luminescence quenching of the metallole-containing polymers by the nitroaromatic analyte. Three common explosives were tested, Trinitrotoluene (TNT), 2,4-dinitrotoluene (DNT), and picric acid (PA). Stock solutions of the explosives were prepared in toluene. Aliquots (1-5 μL) of the stock (containing nanogram-levels of analyte) were syringed onto either Whatman filter paper or a CoorsTek® porcelain spot plate and allowed to dry completely. Solutions of the polymers (0.5-1% w:v) were prepared in acetone (PSi, PGe), 1:1 toluene:acetone (PDEBGe), 2:1 toluene:acetone (PDEBSi), or toluene (PDEBSF). A thin film of a polymer was applied to the substrate by spray coating a polymeric solution onto the substrate and air drying. The coated substrates were placed under a black light to excite the polymer fluorescence. Dark spots in the film indicate luminescence quenching of the polymer by the analyte. The process was carried out for each of the three explosive analytes with each of the six polymers on both substrates.

### Results and Discussion for Nitroaromatics

Nitroaromatic explosives may be visually detected in nanogram quantities by fluorescence quenching of photoluminescent metallole-containing polymers. Detection limits depend on the nitroaromatic analyte as well as on the polymer used.

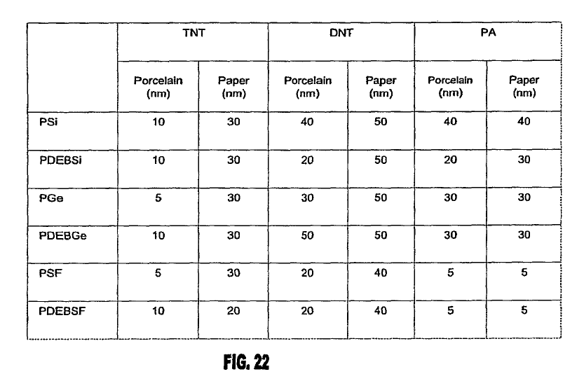

FIG. 22 summarizes the detection limits of TNT, DNT, and picric acid using the five metallole-containing polymers synthesized, PSi, PDEBSi, PGe, PDEBGe, PSF and PDEBSF.

In all cases, the detection limit of the explosives was as low or lower on the porcelain than on paper, likely because the solvated analyte may be carried deep into the fibers of the paper during deposition, thus lowering the surface contamination after solvent evaporation. Less explosive would be present to visibly quench the thin film of polymer on the surface. This situation is less pronounced in actuality when explosives are not deposited via drop-casting from an organic solution, but handled as the solid. Illumination with a black light (λₑₓ˜360 nm) excites the polymer fluorescence near 490-510 nm for the siloles, 470-500 for germoles. The silafluorene luminescence, which peaks at 360 nm, is very weak in the visible region, but it is sufficient for visible quenching.

In testing, the luminescence quenching of three polymers, PSi, PDEBSi, and PGe, by 200, 100, 50, and 10 ng TNT on porcelain plates was observed on a porcelain plate. Also observed was the luminescence quenching of polysilole by each analyte at different surface concentrations.

The method of detection is through electron-transfer luminescence quenching of the polymer luminescence by the nitroaromatic analytes. Consequently, the ability of the polymers to detect the explosives depends on the oxidizing power of the analytes. The oxidation potentials of the analytes follow the order TNT>PA>DNT. Both TNT and PA have three nitro substituents on the aromatic ring which account for their higher oxidizing potential relative to DNT, which has only two nitroaromatic substituents. PA has a lower oxidation potential than TNT due to the electron donating power of the hydroxy substituent. The molecular structure accounts for the lowest detection limit for TNT, followed by PA and DNT.

Luminescence quenching is observed immediately upon illumination. The polymers are photodegradable, however, and luminescence begins to fade after a few minutes of continual UV exposure. Nevertheless, these polymers present an inexpensive and simple method to detect low nanogram level of nitroaromatic explosives.

### Experimental Results and Discussion for Nitramine- and Nitrate Ester-Type Explosives

To determine the ability of the method to detect nitramine- and nitrate ester-type compounds, toluene solutions of RDX (representative of nitramine explosives) and PETN (representative of nitrate ester explosives) were syringed onto filter paper, and allowed to dry, leaving the explosive residue on the surface of the filter paper. The paper was then sprayed with a solution composed of 0.75 M KOH and 2,3-Diaminonaphthalene (DAN) (0.6 mg/mL) in acetone:DMSO:Ethanol (9:2:9). Heat was applied with a standard heat gun for approximately 3 seconds until the paper was dry. A second reagent, composed of 1:1 Ethanol:Phosphoric acid was sprayed on to the paper. A second application of heat was applied for 3 seconds until the paper was dry. The paper was then illuminated with a UV lamp (365 nm), and a bluish-green light appeared over the areas where explosive residue was present, indicating the presence of explosives. Low nanogram levels of RDX and PETN were detected by this method.

The emitted light is due to a chemical reaction between the explosives and applied base, followed by a subsequent reaction with acid. The base attacks the explosive to liberate nitrite. Heat is helpful in driving this reaction. The applied acid then reacts with the nitrite to form nitrous acid, and the reactive nitronium ion. This species reacts with the DAN to form a triazole compound, 1-H-naphthotriazole, which emits bluish-green luminescence upon UV-illumination.

While various embodiments of the present invention have been shown and described, it should be understood that modifications, substitutions, and alternatives are apparent to one of ordinary skill in the art. Such modifications, substitutions, and alternatives can be made without departing from the spirit and scope of the invention, which should be determined from the appended claims.

Various features of the invention are set forth in the appended claims.

What is claimed is:

1. A method for detecting one or more nitrogen-based explosives that may be present in or on a sampling substrate or in an environment to which the sampling substrate has been exposed, said method comprising:
exposing the sampling substrate to a first reagent having a luminescent polymer or copolymer to detect nitroaromatic explosive particulates;
exposing the sampling substrate to a stimulation wavelength;
observing the presence or absence of luminescence to determine the corresponding presence or absence of nitroaromatic explosive particulates;
exposing the sampling substrate to a second reagent capable of both eliminating the luminescence from the polymer of the first reagent, and reacting with nitramine- and nitrate ester-type explosives to release nitrite;
exposing the sampling substrate to a third reagent to react with the nitrite and with a diaminoaromatic, present in one of either the first, second or third reagent to form a luminescent compound;
exposing the sampling substrate to stimulation; and
observing the presence or absence of luminescence to determine the corresponding presence or absence of nitrate ester- or nitramine-based explosives.

2. The method of claim 1 wherein said one of either the first, second or third reagents is selected to include a diaminoaromatic, preferably 2,3-diaminonaphthalene (DAN), to react with the third reagent and nitrite to form a luminescent triazole compound, preferably 1-H-napthotriazole.

3. The method of claim 1 further comprising heating the sampling substrate to a temperature sufficient to effectively release nitrite from nitramine- or nitrate ester-type explosives following the step exposing the sampling substrate to the second reagent.

4. The method of claim 1 further comprising heating the sampling substrate to help speed the chemical reaction and/or solvent evaporation following the step of exposing the substrate to the third reagent.

5. The method of claim 1 wherein said step of observing the presence or absence of luminescence involves visual inspection and/or using artificial sensor, including one of a fluorimeter, a CCD camera, or a visual or ultraviolet camera.

6. The method of claim 1 wherein the steps of exposing the sampling substrate to the first, second and third reagents further comprises spraying the first, second and third reagents onto the sampling substrate.

7. The method of claim 1 wherein all three of the first, second and third reagents are each sprayed onto, or otherwise applied to, predetermined regions of the sampling substrate prior to exposure of the sampling substrate to the environment suspected of being contaminated by explosives.

8. The method of claim 1 wherein all three of the first, second and third reagents are each sprayed onto, or otherwise applied to, predetermined regions of the sampling substrate following exposure of the sampling substrate to the environment suspected of being contaminated by explosives.

9. The method of claim 1 wherein the first reagent contains a metallole polymer.

10. The method of claim 9, wherein the metallole polymer is selected from the group consisting of silole or germole polymers and metallole-containing polymers.

11. The method of claim 10, wherein a metallole-containing polymer is selected from the group consisting of PDEBsilole, PDEBgermole, Polysilafluorene, and Polygermafluorene.

12. The method of claim 1 wherein the luminescent polymer is selected from the group consisting of photoluminescent polyacetylenes, poly(p-phenylenevinylenes), and poly(p-phenyleneethynylenes).

13. The method of claim 1 wherein the first reagent is selected to be a 1 mg/mL solution of Polysilole in a 1:1 acetone:toluene solvent, the second reagent is selected to be a solution of 2,3-diaminonaphthalene (0.6 mg/mL) in a 0.75 M KOH solution in 2:9:9 dimethylsulfoxide:acetone:ethanol solvent, and the third reagent is selected to be a 1:1 solution of phosphoric acid and ethanol.

14. The method of claim 1 wherein the first reagent is selected to be a 0.5 mg/mL Polysilole and 1 mg/mL 2,3-diaminonaphthalene acetone solution, the second reagent is selected to be a 0.75 M KOH solution in 3:2 Ethanol:Dimethylsulfoxide, and the third reagent is selected to be a 1:1 solution of phosphoric acid and ethanol.

15. The method of claim 1 where said sampling substrate is the surface or environment that is suspected of containing trace explosives contamination.

16. A method for detecting one or more nitrogen-based explosives that may be present in a sampling substrate comprising:
determining the presence or absence of nitroaromatic-based explosives using luminescent polymers and copolymers to observe luminescence quenching by the nitroaromatic-based explosives;
eliminating luminescence of the luminescent polymers and copolymers; and
determining the presence or absence of either nitrate ester- or nitramine-based explosives by observing the presence or absence of luminescence of a triazole compound.

17. The method of claim 16, wherein the triazole compound comprises 1-H-naphthotriazole.

18. The method of claim 16 wherein said step of determining the presence or absence of either nitrate ester- or nitramine-based explosives comprises first reacting a base with the explosives believed to be present on the sampling substrate, followed by a further reaction with a diaminoaromatic and an acid.

19. The method of claim 16 where said sampling substrate is the surface or environment that is suspected of containing trace explosives contamination.

20. The method of claim 16 where said luminescent polymer comprises a photoluminescent or electroluminescent polymer.

21. A method of detecting nitramines and nitrate esters believed to be present on a sampling substrate comprising:
exposing the sampling substrate to a first reagent that is formulated to react with nitramine- and nitrate ester type-explosives to release nitrite; and
exposing the sampling substrate to a second reagent that contains an acid to react with the nitrite and a diaminoaromatic, present in either the first or second reagent, to form a triazole that will luminesce.

22. The method of claim 21 wherein the triazole will fluoresce under exposure to a stimulation wavelength.

23. The method of claim 21, wherein the first reagent comprises KOH.

24. The method of claim 21 wherein the diaminoaromatic is selected to be 2,3-diaminonaphthalene (DAN).

25. The method of claim 21 wherein the second reagent is selected to include an acid, such as phosphoric acid.

26. The method of claim 22 where said sampling substrate is the surface or environment that is suspected of containing trace explosives contamination.
